(19) United States
(12) Patent Application Publication
(10) Pub. No.: US 2026/0294886 A1
(43) Pub. Date: Oct. 1, 2026

(54) APPLICATION OF CBL INHIBITOR NX1607 IN PREPARING DRUG FOR TREATING ATHEROSCLEROSIS

(71) Applicant: PEKING UNIVERSITY, Beijing (CN)

(72) Inventors: Xunde XIAN, Beijing (CN); Yitong XU, Beijing (CN); Ling ZHANG, Beijing (CN); Jingxuan CHEN, Beijing (CN); Guolin MIAO, Beijing (CN); Wei HUANG, Beijing (CN); Yuhui WANG, Beijing (CN)

(73) Assignee: PEKING UNIVERSITY, Beijing (CN)

(21) Appl. No.: 19/367,105

(22) Filed: Oct. 23, 2025

Related U.S. Application Data
(63) Continuation of application No. PCT/CN2025/105317, filed on Jun. 30, 2025.

(30) Foreign Application Priority Data
Mar. 28, 2025 (CN) 2025103810526

(51) Int. Cl. A61K 31/454; A61P 9/10

(52) U.S. Cl. CPC A61K 31/454; A61P 9/10

(57) ABSTRACT

Disclosed is an application of a CBL inhibitor in preparing a drug for treating atherosclerosis, where the CBL inhibitor is preferably NX1607 and can inhibit necrotic core formation in plaques in a non-lipid-dependent manner, thereby stabilizing atherosclerotic plaques and delaying disease progression. This disclosure breaks through the limitations of conventional lipid-lowering drugs by directly intervening in the critical pathological process of plaque instability through regulation of the clearance mechanism of apoptotic cells within the inflammatory microenvironment, providing a novel targeted strategy for treating atherosclerosis.

### CROSS-REFERENCE TO RELATED APPLICATIONS

The application claims priority to Chinese patent application No. 2025103810526, filed on Mar. 28, 2025, the entire contents of which are incorporated herein by reference.

### TECHNICAL FIELD

This disclosure relates to the technical field of biomedicine and, in particular, to an application of CBL inhibitor NX1607 in preparing a drug for treating atherosclerosis.

### BACKGROUND

NX1607 is an orally administered CBL inhibitor with a structure of

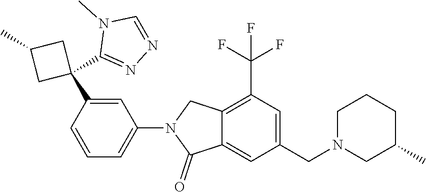

and an IC50 of <1 nM. It has been demonstrated to enhance antigen recall, reduce T cell exhaustion, and increase cytokine production upon T cell receptor stimulation, overcoming inhibitory signals from a tumor microenvironment. As a unique molecular glue, NX1607 is capable of binding to Casitas B-lineage lymphoma proto-oncogene (CBL-B) and promoting its association with various subunits, thereby locking the CBL-B in an inactive conformation and preventing its transition to an active state. This mechanism effectively inhibits the function of CBL-B.

Atherosclerosis (hereinafter referred to as AS) is a vascular disease characterized by chronic inflammation and dysregulated lipid metabolism. Its pathogenesis involves multiple processes, including endothelial injury, deposition of oxidized low-density lipoprotein (ox-LDL), infiltration of monocytes/macrophages, formation of foam cells, and proliferation of smooth muscle cells. Although lipid abnormalities (e.g., elevated LDL and dysfunctional HDL) and inflammatory responses are considered core drivers of AS, existing mainstream therapies (e.g., statins) primarily act by reducing circulating lipid levels and have limited effects on improving plaque stability. Clinical data indicate that even with an intensive lipid-lowering therapy, patients still face a significant residual cardiovascular risk (residual risk rate up to 30%-40%), suggesting that non-lipid-dependent mechanisms (such as dysregulated inflammatory signaling and impaired efferocytosis) play an important role in AS progression.

Currently, only lipid-lowering agents or anti-inflammatory drugs combined with antithrombotic agents are clinically available to delay the progression of AS, but they cannot effectively reverse AS caused by various etiologies. So far, no application of NX1607 in preparing a drug for treating AS has been disclosed in the prior art.

### SUMMARY

To address the aforementioned technical problems, this disclosure provides an application of a CBL inhibitor NX1607 in preparing a drug for treating atherosclerosis. The drug can inhibit necrotic core formation in plaques in a non-lipid-dependent manner, thereby stabilizing atherosclerotic plaques and delaying disease progression. Its mechanism of action is independent of lipid metabolism regulation and primarily exerts anti-AS effects through the following pathways. Compared to conventional lipid-lowering drugs, the innovativeness of NX1607 is reflected in: (1) independence from LDL/HDL metabolism regulation, making it suitable for patients with different lipid levels, particularly high-risk patients with normal lipid levels but unstable plaques; (2) targeting the plaque microenvironment, thereby inhibiting necrotic core formation at its source; and (3) avoiding systemic side effects of broad-spectrum anti-inflammatory drugs through specific inhibition of CBL.

This disclosure provides an application of a CBL inhibitor in preparing a drug for treating atherosclerosis, where the CBL inhibitor is a compound having a chemical structure of Formula I, or a pharmaceutically acceptable salt, a prodrug, or a metabolite thereof:

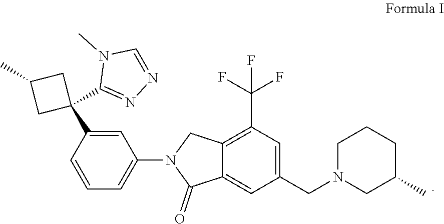

Preferably, the CBL inhibitor is NX1607.

The atherosclerosis is selected from one or more of coronary atherosclerosis, carotid atherosclerosis, cerebral atherosclerosis, aortic atherosclerosis, or lower extremity atherosclerosis, preferably the coronary atherosclerosis or the carotid atherosclerosis.

The atherosclerosis is selected from stable atherosclerotic plaques or unstable atherosclerotic plaques, preferably the unstable atherosclerotic plaques.

The atherosclerosis is further preferably atherosclerosis with normal lipid levels but unstable plaques.

Furthermore, atherosclerosis is clinically classified histologically into eight types. Among these types, Types I-III are early-stage lesions that may regress. Types IV-VI are progressive lesions, where Type IV-V lesions are plaques containing a large necrotic lipid core covered by a fibrous cap, accompanied by minor calcification, which may cause luminal stenosis; and Type VI lesions exhibit surface ulceration, intraplaque hemorrhage, thrombosis, and are further characterized by a thin and irregular fibrous cap. In severe cases, plaque rupture occurs at the shoulder region with the thinnest fibrous cap and the most abundant foam cell infiltration, which is considered vulnerable atherosclerotic plaque. Type VII lesions are purely calcified plaques. Type VIII lesions are fibrous plaques without a lipid core, potentially accompanied by minor calcification. Type VIII lesions may evolve from regression or transformation of lipid components in Type VI lesions. Based on its capability of directly intervening in the critical pathological process of plaque instability through regulation of the clearance mechanism of apoptotic cells within the inflammatory microenvironment, this disclosure targets the plaque microenvironment to inhibit necrotic core formation at its source. Consequently, the drug of this disclosure can be used for treating Types I-VI atherosclerotic lesions, preferably one or more of Types I, II, III, IV, V, or VI.

Simultaneously, this disclosure also provides an application of the aforementioned drug in preparing a drug for inhibiting necrotic core formation in atherosclerotic plaques, preferably by inhibiting necrotic core formation in a non-lipid-dependent manner.

This disclosure further provides an application of the aforementioned drug in preparing a drug for inhibiting lipid accumulation in plaques or reducing inflammatory infiltration.

To facilitate administration, the drug of this disclosure further includes a pharmaceutically acceptable excipient.

The drug of this disclosure is selected from orally administered preparations or parenteral preparations.

Alternatively, the drug of this disclosure is selected from solid preparations, preferably one of capsules, tablets, granules, or pills.

Alternatively, the drug of this disclosure is selected from one of orally administered liquids or emulsions.

Alternatively, the drug of this disclosure is selected from one of injections, lyophilized powder injections, or inhalants.

This disclosure has the following beneficial effects:
1. The drug of this disclosure inhibits necrotic core formation in plaques in a non-lipid-dependent manner, thereby stabilizing atherosclerotic plaques and delaying disease progression. It breaks through the limitations of conventional lipid-lowering drugs by directly intervening in the critical pathological process of plaque instability through regulation of the clearance mechanism of apoptotic cells within the inflammatory microenvironment, providing a novel targeted strategy for treating atherosclerosis.
2. Compared to conventional lipid-lowering drugs, the innovativeness of the CBL inhibitor NX1607 is reflected in: (1) independence from LDL/HDL metabolism regulation, making it suitable for patients with normal lipid levels but unstable plaques; (2) targeting the plaque microenvironment, thereby inhibiting necrotic core formation at its source; and (3) avoiding systemic side effects of broad-spectrum anti-inflammatory drugs through specific inhibition of CBL.

### BRIEF DESCRIPTION OF THE DRAWINGS

FIG. 1 shows a dietary regimen for Ldlr knockout mice, where Ldlr^{-/-} represents the Ldlr knockout mice; a solvent gavage control group received 0.5% sodium carboxymethyl cellulose as a control; and NX1607 represents an NX1607 drug gavage treatment group;

FIG. 2 shows statistical graphs of plasma total cholesterol (TC) and triglyceride (TG) levels in Ldlr^{-/-} knockout mice, where the left graph shows plasma total cholesterol levels, while the right graph shows plasma triglyceride levels; a solvent gavage control group received 0.5% sodium carboxymethyl cellulose as a control; and NX1607 represents an NX1607 drug gavage treatment group;

FIG. 3 shows plasma lipoprotein distribution in Ldlr^{-/-} knockout mice, where VLDL/CM peak represents very low-density lipoprotein and chylomicron levels; LDL represents low-density lipoprotein levels; HDL represents high-density lipoprotein levels; a solvent gavage control group received 0.5% sodium carboxymethyl cellulose as a control; and NX1607 represents an NX1607 drug gavage treatment group;

FIG. 4 shows plasma alanine aminotransferase and aspartate aminotransferase levels in Ldlr^{-/-} knockout mice, where a solvent gavage control group received 0.5% sodium carboxymethyl cellulose as a control; and NX1607 represents an NX1607 drug gavage treatment group;

FIG. 5 shows inflammation-related results from complete blood count in Ldlr^{-/-} knockout mice, including white blood cell count (WBC), neutrophil count (Neu), lymphocyte count (Lym), and monocyte count (Mon), where a solvent gavage control group received 0.5% sodium carboxymethyl cellulose as a control; and NX1607 represents an NX1607 drug gavage treatment group;

FIG. 6 shows erythrocyte-related results from complete blood count in Ldlr^{-/-} knockout mice, including red blood cell count (RBC), hemoglobin concentration (HGB), hematocrit (HCT), mean corpuscular volume (MCV), mean corpuscular hemoglobin (MCH), and mean corpuscular hemoglobin concentration (MCHC), where a solvent gavage control group received 0.5% sodium carboxymethyl cellulose as a control; and NX1607 represents an NX1607 drug gavage treatment group;

FIG. 7 shows platelet-related results from complete blood count in Ldlr^{-/-} knockout mice, including platelet count (PLT), platelet distribution width (PDW), mean platelet volume (MPV), and plateletcrit (PCT), where a solvent gavage control group received 0.5% sodium carboxymethyl cellulose as a control; and NX1607 represents an NX1607 drug gavage treatment group;

FIG. 8 shows gross Oil Red O staining of blood vessels in Ldlr^{-/-} knockout mice, where a solvent gavage control group received 0.5% sodium carboxymethyl cellulose as a control; and NX1607 represents an NX1607 drug gavage treatment group; and

FIG. 9 shows HE staining, Oil Red O staining, CD68 staining, and TUNEL staining of outflow tracts in Ldlr^{-/-} knockout mice, where a scale bar of 100 μm was adopted; a solvent gavage control group received 0.5% sodium carboxymethyl cellulose as a control; and NX1607 represents an NX1607 drug gavage treatment group.

### DETAILED DESCRIPTION OF THE EMBODIMENTS

The specific embodiments of this disclosure will be further described below with reference to the accompanying drawings. It should be noted that the description of these embodiments is intended to facilitate understanding of this disclosure, but does not constitute a limitation thereon.

Furthermore, the technical features involved in various embodiments of this disclosure described below can be combined with each other as long as they do not conflict with each other.

### I. Experimental Animals and Their Husbandry

Species, sex, age, and source of experimental animals: Ldlr gene (gene ID: 16835; gene function: involved in lipid transport; regulation of inflammatory response; and regulation of lipid metabolic processes), male, 8 weeks old, body weight: 19-25 g. Ldlr knockout mice (Ldlr^{-/-}) were purchased from GemPharmatech (Nanjing, China).

### II. Diet Formula for Experimental Animals

Western Diet (WD) purchased from Research Diets, Inc., product number: D12108C.

### III. Animal Husbandry and Grouping

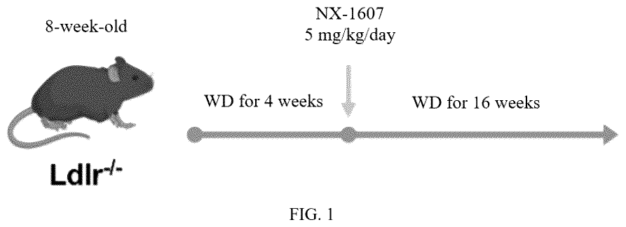

All experiments were performed in accordance with the Guide for the Care and Use of Laboratory Animals (National Institutes of Health (NIH) Publication No. 85Y23, revised 1996) and approved by the Institutional Animal Care and Use Committee of Peking University (LA2023460). All animal breeding and husbandry were performed in the SPF-level animal facility of Peking University Health Science Center (License No.: SYXK (Jing) 2022-0037). A 12-hour light/dark cycle was maintained, with temperature at 25±2° C. and humidity at 40±5%. Unless otherwise specified, all mice had free access to food and water. As shown in FIG. 1, which illustrates a dosing and dietary regimen for Ldlr knockout mice, 8-week-old mice on a chow diet (CD) were fed the aforementioned Western Diet (WD) for 4 weeks, followed by 16 weeks of NX1607 gavage treatment (NX1607 dosage: 5 mg/kg body weight/day). The mice were divided into two groups: a solvent control group and a drug administration group. The solvent used was 0.5% sodium carboxymethyl cellulose.

### IV. Measurement of Plasma Total Cholesterol and Triglyceride Levels

After the 8-week-old mice on the chow diet (CD) were fed the aforementioned Western Diet (WD) for 4 weeks, followed by 16 weeks of solvent control or NX1607 gavage treatment, the mice were fasted for 4 hours. Blood samples were collected from the orbital vein using anticoagulant or non-anticoagulant tubes. Plasma was separated by centrifugation at 4° C. for 10 minutes. Plasma total cholesterol (TC) and triglyceride (TG) concentrations were measured using commercially available kits from BIOSINO (Beijing, China).

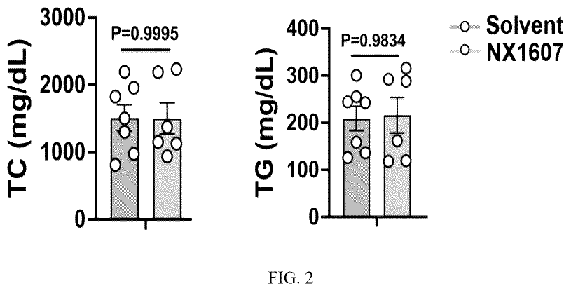

The results, as shown in FIG. 2, indicate that NX1607 treatment had no effect on plasma total cholesterol and triglyceride levels in mice.

### V. Determination of Plasma Lipoprotein Distribution by Fast Protein Liquid Chromatography

After the 8-week-old mice on the chow diet (CD) were fed the aforementioned Western Diet (WD) for 4 weeks, followed by 16 weeks of NX1607 gavage treatment, the mice were fasted for 4 hours. Blood samples were collected from the orbital vein using anticoagulant or non-anticoagulant tubes. Plasma was separated by centrifugation at 4° C. for 10 minutes. Lipoprotein distribution was determined using a fast protein liquid chromatography system. Total cholesterol (TC) and triglyceride (TG) levels in the samples were measured using commercially available kits from BIOSINO (Beijing, China).

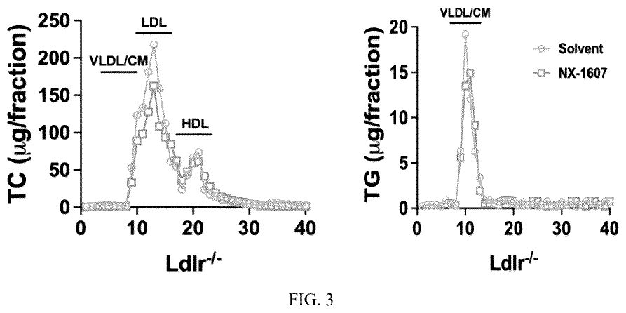

The results, as shown in FIG. 3, indicate that NX1607 treatment had no significant effect on the distribution of various lipoproteins in mouse plasma.

### VI. Measurement of Plasma Alanine Aminotransferase and Aspartate Aminotransferase Levels

After the 8-week-old mice on the chow diet (CD) were fed the aforementioned Western Diet (WD) for 4 weeks, followed by 16 weeks of NX1607 gavage treatment, the mice were fasted for 4 hours. Blood samples were collected from the orbital vein using anticoagulant or non-anticoagulant tubes. Plasma was separated by centrifugation at 4° C. for 10 minutes. Plasma alanine aminotransferase (ALT) and aspartate aminotransferase (AST) levels were measured using commercially available kits from Nanjing Jiancheng Bioengineering Institute.

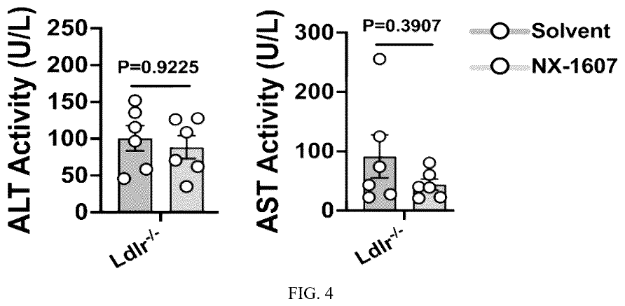

The results, as shown in FIG. 4, indicate that NX1607 treatment had no significant effect on plasma ALT or AST levels in mice. This demonstrates that the use of this drug does not cause hepatotoxicity.

### VII. Measurement of Complete Blood Count in Mice

After the 8-week-old mice on the chow diet (CD) were fed the aforementioned Western Diet (WD) for 4 weeks, followed by 16 weeks of NX1607 gavage treatment, blood samples were collected from the orbital vein using anticoagulant tubes. The blood samples were thoroughly mixed with anticoagulant or diluent immediately upon collection or shortly thereafter for analysis. The mouse blood samples were measured and analyzed using a veterinary hematology analyzer from Beijing Furui Runze Biotechnology Co., Ltd.

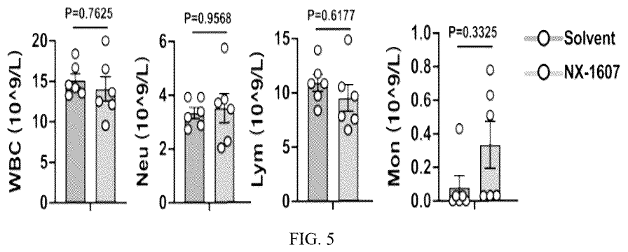

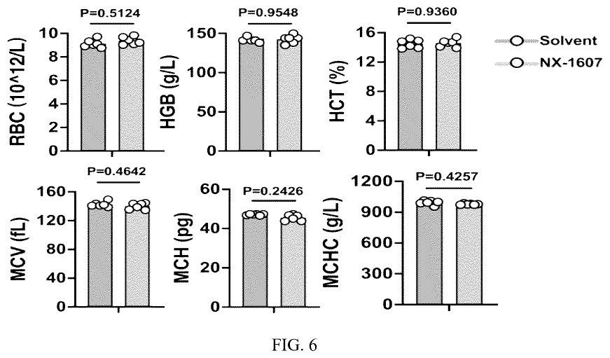

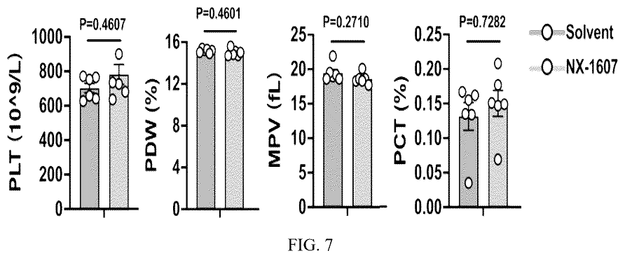

The results, as shown in FIGS. 5-7, indicate that NX1607 treatment had no significant effect on inflammation-related indicators in mouse blood (white blood cells, neutrophils, lymphocytes, and monocytes) (FIG. 5); no significant effect on erythrocyte-related indicators (red blood cells, hemoglobin concentration, hematocrit, mean corpuscular volume, mean corpuscular hemoglobin, and mean corpuscular hemoglobin concentration) (FIG. 6); and no significant effect on platelet-related indicators (platelet count, platelet distribution width, mean platelet volume, and plateletcrit) (FIG. 7). The results from FIGS. 5-7 demonstrate that NX1607 has no adverse effects on complete blood biochemical parameters.

### VIII. Gross Oil Red O Staining of Blood Vessels

After the 8-week-old mice on the chow diet (CD) were fed the aforementioned Western Diet (WD) for 4 weeks, followed by 16 weeks of NX1607 gavage treatment, the entire aorta was harvested. The tissue was fixed with paraformaldehyde, dehydrated with 20% sucrose, and stained with Oil Red O.

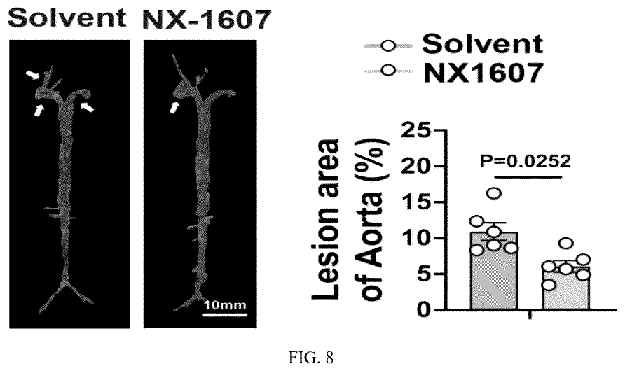

The results, as shown in FIG. 8, indicate that gross Oil Red O staining of the aorta revealed reduced lipid accumulation (red areas) in the NX1607 treatment group, suggesting a significant reduction in aortic plaques. These pathological results demonstrate that NX1607 treatment significantly reduces atherosclerotic plaques in the aorta, indicating a significant therapeutic effect on atherosclerosis.

### IX. HE Staining, Oil Red O Staining, Bodipy Staining, and CD68 Staining of Aortic Outflow Tract

After the 8-week-old mice on the chow diet (CD) were fed the aforementioned Western Diet (WD) for 4 weeks, followed by 16 weeks of NX1607 gavage treatment, the aortic outflow tract (aortic root) was harvested. Cryosections of the aortic root were prepared at a thickness of 7 μm and subjected to HE staining, Oil Red O staining, as well as CD68 and TUNEL immunofluorescence staining.

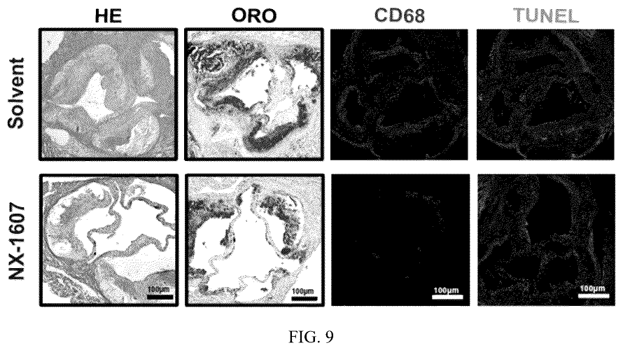

The results, as shown in FIG. 9, indicate that compared to the control group,

NX1607-treated mice showed reduced lipid accumulation in the aortic outflow tract plaques (red areas in ORO staining), suggesting a significant reduction in aortic root plaques; HE- and TUNEL-positive signal area (green areas) demonstrated a significant reduction in necrotic core area in the aortic root; and CD68-positive signal area indicated reduced inflammatory infiltration in the plaque regions of the aortic root.

These pathological results collectively demonstrate that NX1607 treatment significantly reduces atherosclerotic plaques, necrotic areas, and inflammation, indicating a significant therapeutic effect on atherosclerosis.

In conclusion, CBL inhibitors, represented by NX1607, have therapeutic effects on atherosclerosis and can be applied in preparing a drug for treating atherosclerosis.

What is claimed is:

1. An application of a CBL inhibitor in preparing a drug for treating atherosclerosis, wherein the CBL inhibitor is a compound having a chemical structure of Formula I or a pharmaceutically acceptable salt thereof:

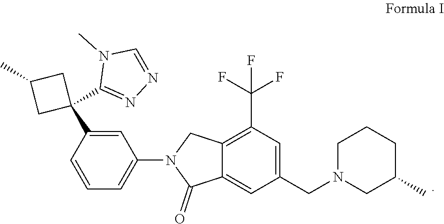

2. The application of claim 1, wherein the atherosclerosis is selected from one or more of coronary atherosclerosis, carotid atherosclerosis, cerebral atherosclerosis, aortic atherosclerosis, or lower extremity atherosclerosis.

3. The application of claim 2, wherein the atherosclerosis is selected from stable atherosclerotic plaques or unstable atherosclerotic plaques.

4. The application of claim 1, wherein the atherosclerosis is selected from one of Type I, II, III, IV, V, or VI atherosclerosis.

5. The application of claim 1, wherein the drug further comprises a pharmaceutically acceptable excipient.

6. The application of claim 1, wherein the drug is selected from orally administered preparations and/or parenteral preparations.

7. The application of claim 1, wherein the drug is selected from solid preparations, comprising one of capsules, tablets, granules, or pills.

8. The application of claim 1, wherein the drug is selected from one of orally administered liquids or emulsions.

9. The application of claim 1, wherein the drug is selected from one of injections, lyophilized powder injections, or inhalants.
